(19) United States
(12) Patent Application Publication
(10) Pub. No.: US 2009/0281462 A1
(43) Pub. Date: Nov. 12, 2009

(54) Device and method for following the movement of a living being

(75) Inventors: Rodolphe Heliot, Grenoble (FR); Christine Azevedo-Coste, Montbazin (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: COMMISSARIAT A L' ENERGIE ATOMIQUE, Paris (FR)

(21) Appl. No.: 12/513,986

(22) PCT Filed: Nov. 13, 2007
(86) PCT No.: PCT/EP07/62280

§ 371 Date: May 7, 2009

(30) Foreign Application Priority Data
Nov. 15, 2006 (FR) 06 54922

(51) Int. Cl. A61B 5/103

(52) U.S. Cl. 600/595

(57) ABSTRACT

A system including a sensor measuring a position of movement, an artificial controller converting the signals from the sensor into state variables of full movement, correlated with a reference model, by performing continual adjustments for estimating these variables directly drawn from the signals of the sensor by results drawn from the reference model. The control which is then provided for completing the movement has good synchronization with the portion accomplished without assistance from the system. Such a system may find application for reproducing or completing the walking of a person with a disabled leg.

The subject of this invention is the following of the movement of a living being in order to reproduce it or complete it; an application which is contemplated today, but which is not exclusive since many other ones are to be contemplated, is providing assistance to walking of a hemiplegic person and therefore only having a single good leg, or of a person having a leg prosthesis.

One then seeks to have the disabled leg perform a movement completing that of the good leg in order to provide a gait as normal as possible. An artificial control of the movement of a disabled leg may assume several forms: in hemiplegic patients, a functional electric stimulation may be produced on the muscles of the disabled leg by means of electrodes in order to have them contract and thereby generate the movement; on a prosthesis, a regulation of the movement of the artificial knee may either be accomplished by blocking it in the phases when the person who is equipped with it, bears upon it, or on the contrary, by leaving it very flexible in the phases where it should not slow down the movement.

It is then tempting to use movement sensors on the good leg and to suitably process their signals for obtaining instructions for controlling the artificial device placed on the disabled leg. The articles of Williamson (Williamson, R. p.; Andrews, B. J.; Au, R.: “Control of neural prostheses. II. Event detection using machine learning “Proceedings of the RESNA” '96 Annual Conference Exploring New Horizons . . . Pioneering the 21^{st} Century, p. (291-3), 1996) and Pappas (Pappas I. P. I.; Keller, T.; Mangold, S.: “A reliable, gyroscope base gait phase detection sensor embedded in a shoe insole” Proceedings of IEEE Sensors 002. First IEEE International Conference on Sensors, p. 1085-8 Vol.2, 2002) indicate a few procedures. Discrete walking states are detected on the signals from sensors and make it possible to provide a control of the disabled leg, but not to perfectly coordinate the movements of both legs. An analogous prior art appears in US-A- 2004/088057. Numerical models for generating a cyclic movement have also been proposed, especially in the field of robotics, in order to for example reproduce a gait. With these studies designed for animating artificial bodies, it is not possible to coordinate or to synchronize satisfactorily a natural limb or an artificial limb in variable gait situations.

The object of the invention is therefore to enhance the existing methods for obtaining better coordination or synchronization of the movements of both legs. It resorts to a numerical model recorded beforehand of the gait, but which is continually adapted to the actual gait by the controller generating the gait on the artificial leg, by means of a digital observer which follows both the model and the signals of the sensors, which are interpreted for inferring the controls to be applied.

An aspect of the invention is a device for following the movement of a living being, comprising a sensor attached to the living being and collecting signals representative of the movement thereof, and an artificial controller for processing the signals from the sensor, wherein the artificial controller comprises a computation module characterized in that the computation model comprises a digital observer capable of delivering an output signal according to a reference model of the recorded movement in the artificial controller, the output signal being a function of the signals of the sensor and of the reference model, the reference model and the output signal being expressed in state variables of the movement relative to a phase of the movement.

Another aspect of the invention relates to a method for following the movement of the living being capable of only accomplishing an effective portion of the movement, in a method for following the movement of a living being, consisting of continuously measuring an effective portion of the movement by a sensor attached on the living being, of using signals from the sensor in order to estimate values of state variables of the movement for producing control instructions and driving a device accomplishing a complementary portion of the movement, characterized in that it consists of also using a reference model, obtained in a calibration step and defined by series of values taken by state variables of the movement in phases of the movement, in order to estimate the values of the state variables of the movement.

A good way for numerically solving the adjustment is obtained if the digital observer continuously carries out an adjustment of a parameter according to the measurements of the sensor by digitally solving a differential equation, and an adjustment of at least one of the state variables according to the intermediate parameter.

In this particular case for restoring walking, the sensor being attached to the thigh of the good leg, the effective portion of the movement being accomplished by the good leg, a good solution is represented if the state variables are an angular velocity and an angular position of the thigh bearing the sensor. A possible set-up of the device is achieved if the digital filter solves a system of equations

$$\{ \begin{matrix}{\hat{x}}_{1} = {x}_{2} \\ {\hat{x}}_{2} = {f}_{1} ( {x}_{1} , {x}_{2} )\end{matrix}$$

in order to generate the reference model of the movement, and the digital observer solves a system of equations ż=f₂(z, u) and then xᵢ=f3 (z, u) wherein z is the intermediate parameter which depends on xᵢ and w₂ the state variables, xᵢ is one of the state variables, u the signal from the sensor (1) and f₁, f₂ and f₃ being functions.

These aspects of the invention as well as other aspects will now be described in connection with the following figures:

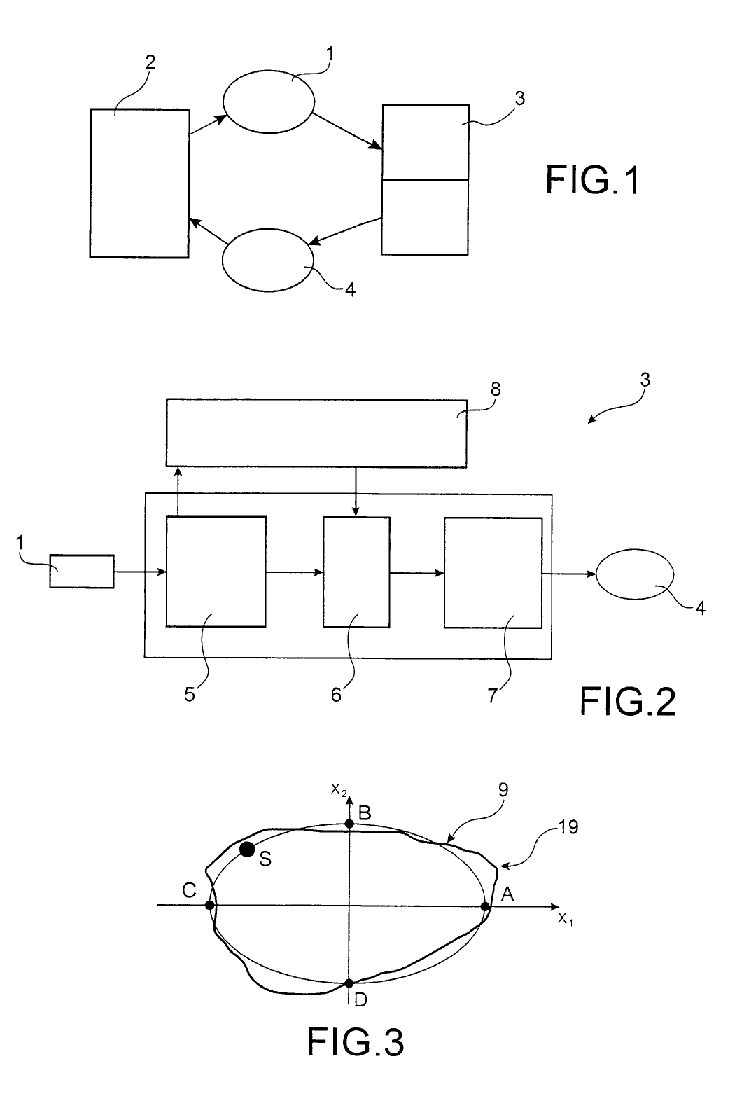

FIG. 1 illustrates the implantation diagram of the method,

FIG. 2 illustrates the artificial controller,

FIG. 3 illustrates the representations of a cycle of movements,

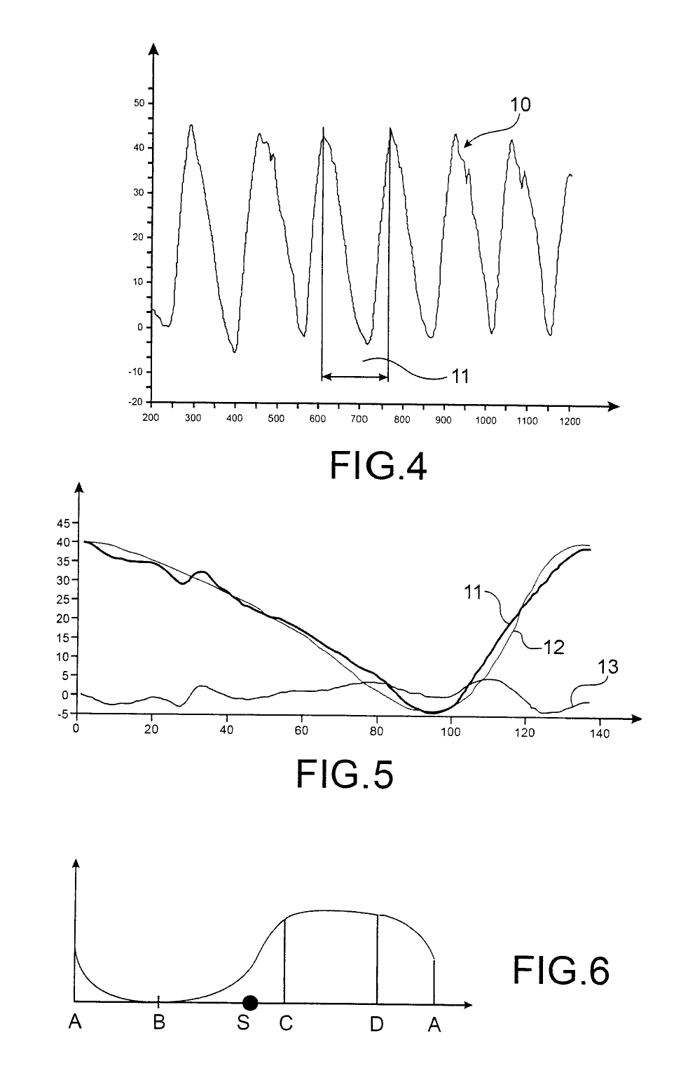

FIG. 4 illustrates the signals from the sensor,

FIG. 5 illustrates the adjustment of the model and experimental results,

and FIG. 6 gives a control function.

We proceed with describing a specific embodiment of the invention for a particular application.

The implantation diagram of the method is summarized in FIG. 1. It comprises at least one sensor, attached on a person intended to be assisted by the method and which is sensitive to the movements of his/her musculoskeletal system 2. The system further comprises an artificial controller 3 which may also be worn by the person, and a driving device 4. The sensor 1 may notably include accelerometers, inclinometers, gyrometers, etc., measuring a direction or a tilt; it may be attached to the thigh or the good leg of the wearer in the case when it is the gait with the method should improve, but other sensors may be placed elsewhere, for example on the trunk of the wearer. The number of sensors 1 is not critical, and a single one is often sufficient.

The artificial controller 3 reacts to measurements of the sensor 1 in order to compute the movement to be applied to the disabled leg; it estimates the instantaneous values taken by the state variables of the movement from measurements of the sensor 1 and elaborates the control to be applied to the driving device 4.

The description now more specifically deals with the artificial controller 3 by means of FIG. 2. The artificial controller 3 comprises three in-line modules: a module 5 for acquiring and digitizing the signal from the sensor 1; a real-time computation module 6 and a module 7 for elaborating the control 7 which provides the control instructions to the driving device 4. The artificial controller 3 may be connected to a microcomputer 8. The latter receives digitized signals stemming from the acquisition module 5 and gives the observer parameters back to the computation module 6, in the way which will be described later on. The acquisition module 5 receives analog signals from the sensor 1, also sends digitized signals to the computation module 6, which itself provides instantaneous values of the state variables of the movement to the control module 7.

Upon applying the method according to the present invention, two main phases are distinguished. The first phase is a calibration phase intended to parameterize the real-time computation module 6 and optionally the module for elaborating the control 7. The calibration phase is broken down into four steps described hereafter. The second phase is a phase of use, during which the real-time computation module 6 is operational.

The present invention is aimed at following a <<cyclic>> movement, such as walking. The signal detected by each sensor used should be cyclic, in other words substantially periodic.

During an initial step of the calibration phase, the signal measured by the sensor is recorded for a duration at least equal to one cycle of the detected signal. A portion of the recorded signal with a duration of one cycle, called a reference signal hereafter, is then selected.

An oscillator type model is then defined, with which a signal may be generated which has a shape substantially identical to the reference signal on a given cycle

The oscillator model Σ used in this embodiment of the present invention belongs to the class of the Lure systems. More specifically, the oscillator is defined from a set of equations using state variables, according to the following formulae:

$$Σ \text{:} \{ \begin{matrix}\dot{x} = A \cdot x + f ( y ) \\ y = C \cdot x \end{matrix}$$

wherein x is a state variable vector, \dot{x} is the x derivative vector, y is a variable output vector, f( ) is a non-linear function and A and C are linear matrices of parameters.

The definition of the f function and of the linear matrices parameters is achieved by an optimization method.

Once the model Σ is adjusted, an observer system Σ′ associated with the model Σ is defined according to the following system of equations:

$${Σ}^{′} \text{:} \{ \begin{matrix}{x}^{\hat{.}} = A \cdot \hat{x} + f ( y ) + K ( \hat{y} - y ) \\ \hat{y} = C \cdot \hat{x} \end{matrix}$$

Wherein \hat{x} is an estimate of vector x, ŷ is an estimate of the output vector y, and K is a linear matrix of parameters.

The definition of K is achieved depending on the selection of A, C and f according to a method known to one skilled in the art. Examples of definitions are given subsequently. The observer system therefore differs from the model by an adaptive term K(ŷ−y) which, as this will be seen, provides correction of the control when the gait of the good leg differs from the model so as to improve coordination and synchronization of the legs.

A control function w is then defined, intended to be applied by the model for elaborating the control 7. The control instructions D depend on values of estimated state variables \hat{x} according to the formula D=w(\hat{x}).

Once defined, the observer system Σ′ and the control function w are respectively transferred, or in other words programmed into the real-time computation module 6 and into the module 7 for elaborating the control.

Once this calibration phase is completed, the microcomputer 8 may be disconnected. From then on, the programmed artificial controller 3 may operate in a standalone way.

When using the artificial controller 3, the real-time computation module 6 receives the signal measured by the sensor after digitization. The computation module 6 is an observer system corresponding to the system Σ′ defined beforehand, in which the input y is now replaced with the measurement u of the sensor. The observer system of the computation module 6 may therefore be written down according to the following formula:

$${Σ}^{′} \text{:} \{ \begin{matrix}{x}^{\hat{.}} = A \cdot \hat{x} + f ( u ) + K ( \hat{y} - u ) \\ \hat{y} = C \cdot \hat{x} \end{matrix}$$

A detailed exemplary application of the present invention is described hereafter.

Reference is made to FIG. 3. A movement may be defined by state variables; two of them (x1 and x2) may be sufficient in the case of walking, i.e. the angular position and the angular velocity of the thigh. A full stride, corresponding to two consecutive steps is a cycle of the movement which is illustrated by a closed model curve 9, each point of which or each phase of the stride is defined by instantaneous values of x1 and x2. The model curve 9 corresponds to the cycle which the state variables \hat{x} would follow, as estimated by the real-time computation module 6 if the measurement signal u received by the latter matched the reference signal defined above.

In actual walking, each of the strides will be different, but only deviating from the model by small amounts. The signal from the sensor 1 is approximately periodic and may assume the aspect illustrated in FIG. 4, where a measurement curve 10 shows its intensity versus time.

The description now more specifically deals with the computation module 6. It is used for converting the signals from the sensor 1 and shaped by the acquisition module 5 into state variables of the model retained for walking.

The signal from the sensor 1 is digitized by the module 5 with a given sampling frequency. The module 5 provides a succession or series of measurement samples u(n), where n corresponds to a given instant. For each sample u(n), the computation module 6 determines the values \hat{x} (n) of the estimated state values. Each \hat{x} (n) value is not only a function of the present sample u(n) but also of the history of the previous samples.

Referring to FIG. 3, each value \hat{x} (n) of the estimated state variables may be represented by a point of the plane. A curve 19 represents an exemplary sequence of values of estimated state variables, obtained for an <<imperfect>> stride. With the system Σ′, it is possible to ensure convergence of the curve 19 towards the model curve 9.

In the particular case of the invention applied to walking, the element Σ may be defined by means of the system of equations (3):

$$Σ \{ \begin{matrix}{\dot{x}}_{1} = {x}_{2} \\ {\dot{x}}_{2} = μ ( 1 - {bx}_{1} - {x}_{1}^{2} ) {x}_{2} - {ω}_{0}^{2} {x}_{1} \\ y = {x}_{1} \end{matrix}$$

wherein μ, b and ω₀ are optimally adjusted by the microcomputer 8 in order to minimize the error between the results of computations and the measurements during the preliminary calibration step. FIG. 5 gives, as a superposition to the reference profile 11 illustrated in FIG. 4, but with a different scale, the curve of the output (y) 12 of the element Σ and the error curve 13 between curves 11 and 12. In order to determine the system Σ′, let us consider the variable z defined by equation (4) z=x₂+k₁.y+k₂.y²+k₃.y³. Equation (5) is obtained by utilizing the system of equations (3):

ż=(μ+k₁)x₂+(2.k₂−μ.b)x₁.x₂+(3.k₃−μ)x₁²−x₂−ω₀².x₁

By selecting k₁=−μ−1, k₂=μ.b/2 and k₃=μ/3, equation (6) is obtained:

ż=−z+(K₁−ω₀²)y+k₂.y²+k₃.y³

which, with the system of equations (7):

$$\{ \begin{matrix}{\hat{x}}_{1} = y \\ {\hat{x}}_{2} = z - {k}_{1} \cdot y - {k}_{2} \cdot {y}^{2} - {k}_{3} \cdot {y}^{3} \\ \hat{y} = {x}_{1} \end{matrix}$$

corresponds to the observer system Σ′. The coefficients k₁, k₂ and k₃ are obtained from values b, μ and ω₀ computed by the element Σ in the calibration step.

When using the artificial controller 3, the computation module 6 receives the measurement signal u. The computation module 6 is then an observer system corresponding to the system Σ′ defined beforehand, in which the input is now replaced therein with the measurement u from the sensor.

The element Σ′ again continually calculates the value of z after having calculated ż according to equation (6), and then \dot{x}₂ according to the second equation of the system (7) and delivers at the output, values of the state variables \hat{x} (\hat{x}₁, \hat{x}₂).

In the present case, \hat{x}₂ corresponds to an estimation of the angular velocity and \hat{x}₁ to an estimation of the angular position.

We emphasize that the element Σ, used during the calibration step for providing the values of the coefficients and building the model providing the relationships between the state variables, remains inactive during the use of the invention when only the element Σ′ is working, while the latter was inert during the calibration. The microcomputer 8 is moreover removed during this use, after having provided the results to the artificial controller 3.

The element Σ is used for describing the movement, the element Σ′ for synchronizing the control with it.

Such is the system by which the state variables of the movement are continually computed, with good synchronization with the indications of the sensor 1. It should be noted that perfect restoration of the state variables would be obtained only in the case when the gait would exactly corresponds to the model curve 9 for each of the strides, which is not the case in reality, but another effect of the observation element Σ′ is to mitigate the errors originating from the difference between the actual strides and the modeled stride.

The control module 7 delivers control instructions C depending on estimated state variables \hat{x} (\hat{x}₁, \hat{x}₂). According to formulae such as D=w(\hat{x}). The function w depends i.a. on the driving device 4 placed on the disabled limb and on the nature of the control which has to be applied to it.

In order to define the type of function w, which may be applied for hemiplegic persons, the following experimental procedure may for example be used: on an able-bodied person (other than the disabled person, for which assistance is desired subsequently), a movement sensor is placed on one of his/her limbs corresponding to the limb of the disabled person on which the movement sensor will be placed subsequently, as well as a muscular activity sensor on the other of his/her limbs. At least one stride of the able-bodied person is then observed. For a measurement cycle, a correspondence is established between the estimated variable state values (from the corresponding measurement signal) and the measurements of muscular activity detected by the muscular activity sensor. Moreover, a correspondence may be defined between each measurement of muscular activity of the good limb of the able-bodied person and a value of stimulation parameters of the disabled limb of the disabled person, with which the corresponding muscular activity measurement may again be found. Thus, the function w may be defined, for example, as a correspondence table associating with each estimated state variable value \hat{x}, a control corresponding to the aforementioned stimulation parameters.

The control is performed after each phase of the cycle. FIG. 6 gives a practical example according to the cycle of FIG. 3, for points corresponding to A, B, C or D of this group and any point S.

Moreover, in order to take differences between individuals into account, the aforementioned function w may be parameterized so as to be able to notably adjust the amplitude of the movements of the disabled person. The definition of such parameters may then be achieved during the calibration phase.

According to an alternative embodiment of the device described above and illustrated in FIG. 2, the artificial controller 3 does not contain any digitization module. The signal received by the real-time computation module 6 is then analog. The computation module 6 and the module 7 are then analog devices.

The main advantages of the invention are therefore the continuous character of the control which results in much better synchronization, stability of the oscillator, and ease of application.

The method was described in the case of a disabled person and more particularly a hemiplegic person. Other applications are possible.

For example, a robot may be controlled, possibly by remote operation, from controls emitted by the module 7. This robot may for example follow the person on which the sensor 1 is placed in order to assist him/her in these movements.

1-16. (canceled)

17. A device for following movement of a living being, comprising:
a sensor attached to the living being and collecting signals representative of the movement thereof; and
an artificial controller configured to process signals from the sensor, wherein the artificial controller comprises a computation module, and the computation module comprises a digital observer configured to deliver an output signal according to a reference model of the movement recorded in the artificial controller, the output signal being a function of the signals from the sensor and of the reference model, the reference model and the output signal being expressed as state variables of the movement relative to a phase of the movement.

18. The device for following movement according to claim 17, further comprising a calibration computer configured to deliver parameters of the reference model to the computation module depending at least on a preliminary series of signals from the sensor.

19. The device for following movement according to claim 18, wherein the calibration computer is separable from the artificial controller.

20. The device for following movement according to claim 18, wherein the calibration computer includes a digital filter for the preliminary series of signals from the sensor.

21. The device for following movement according to claim 20, wherein the digital filter is compliant with an oscillator according to a Lure system.

22. The device for following movement according to claim 17, wherein the sensor measures an angular velocity and an angular position.

23. The device for following movement according to claim 17, further comprising a module for elaborating a control for completing or reproducing the movement by a driving device.

24. A method for following movement of a living being, comprising:
continuously measuring an effective portion of the movement by a sensor attached on the living being;
using signals from the sensor to estimate values of state variables of the movement to produce control instructions and to drive a device accomplishing a complementary portion of the movement; and
using a reference model, obtained in a calibration and defined by a series of values taken by the state variables of the movement in phases of the movement, to estimate the values of the state variables of the movement.

25. The method for following movement according to claim 24, wherein the state variables comprise two state variables that may be inferred from each other by time derivation.

26. The method for following movement according to claim 25, wherein the state variables represent a position and a velocity.

27. The method for following movement according to claim 24, wherein a digital observer continuously carries out an adjustment of an intermediate parameter according to the measurements of the sensor by digitally solving a differential equation and an adjustment of at least one of the state variables according to the intermediate parameter.

28. The method for following movement according to claim 26, wherein the movement is walking.

29. The method for following movement according to claim 28, wherein the state variables are an angular velocity and an angular position of a thigh bearing the sensor.

30. The method for assisting movement according to claim 29, wherein the digital filter solves a system of equations
$$\{ \begin{matrix}{\hat{x}}_{1} = {x}_{2} \\ {\hat{x}}_{2} = {f}_{1} ( {x}_{1} , {x}_{2} )\end{matrix}$$
in order to generate the reference model of the movement, and the digital observer solves a system of equations ż=f₂(z, u) and then xᵢ=f3 (z, u), wherein z is the intermediate parameter which depends on xᵢ, x₁ and x₂ the state variables, xᵢ being one of the state variables, u the signal from the sensor, and f₁, f₂ and f₃ being functions.

31. The method for following movement according to claim 24, wherein the reference model is obtained by a digital filter capable of generating a filtered movement cycle, by adjusting parameters of the digital filter for adjusting the filtered movement cycle onto a movement cycle measured by the sensor, the filtered movement cycle becoming the reference model.

32. The method for following movement according to claim 31, wherein a digital observer of the digital filter is used.
